(19) United States
(12) Patent Application Publication
(10) Pub. No.: US 2025/0136540 A1
(43) Pub. Date: May 1, 2025

(54) METHODS FOR EXTRACTING SPERMIDINE FROM RICE GERM

(71) Applicant: NUTRALAND USA, INC., Irvine, CA (US)

(72) Inventors: Ling LIN, Irvine, CA (US); Sanying XU, Irvine, CA (US)

(21) Appl. No.: 18/494,213

(22) Filed: Oct. 25, 2023

(51) Int. Cl. C07C 211/09

(52) U.S. Cl. CPC C07C 211/09

(57) ABSTRACT

Embodiments of the present disclosure relate to methods for extracting and purifying spermidine from rice germ. Such methods may include extracting spermidine from the rice germ with a first solvent; filtering the extraction solution; concentrating the filtrate to obtain coarse extract; optionally mixing the coarse extract with citric acid; extracting with a second solvent; filtering the second extraction solution to remove insoluble matter, and concentrating the filtrate to obtain a rice germ extract. Such methods may produce an extract having a spermidine content of about 1% to about 5% and a total extraction yield of about 80% or more. Compositions of the spermidine extract obtained from the methods are also provided.

### BACKGROUND

### Field

The present disclosure relates to the field of technology to extract spermidine from a natural source. More specifically, the present disclosure relates to methods for extracting spermidine from rice germ.

### Description of the Related Art

Rice germ accounts for 3% of the weight and 50% of the nutritional value of rice grain. Rice germ includes rich protein and starch, dietary fiber, various vitamins, and bioactive substances.

Spermidine is a natural component of rice germ. Research increasingly shows that spermidine can promote autophagy in the body, alongside other benefits, such as anti-aging, improving immunity and stem cell function, protecting the heart and nerves, and anti-inflammatory functions. Therefore, spermidine supplements may be desirable for health and longevity.

Spermidine is a polyamine in rice germ and wheat germ. Polyamines usually include putrescine, spermidine, and spermine, among which the content of spermidine is higher than other types of natural polyamines. spermidine was initially extracted and separated from semen and plays a crucial role in cellular metabolism. Spermidine is not only present in semen but also widely distributed in other tissues and cells in the body.

There are two main sources from which humans obtain spermidine. Madeo F, et al., Spermidine in health and disease, Science, 2018 Jan. 26; 359 (6374):eaan 2788. The first source is food. Spermidine can be extracted from wheat germ, rice germ, soybeans, and mushrooms. Spermidine obtained through food may be quickly absorbed or distributed in the intestines without degradation. Such path of absorption and distribution leads to various concentrations of spermidine in the blood. For example, the Mediterranean diet can cause high concentration of spermidine in the blood. The second source from intestinal microorganisms, which can synthesize spermidine that is then absorbed by the host. For example, bifidobacteria in the intestines can produce spermidine.

Recently, the relationship between spermidine intake and mortality has been analyzed in detail. One study indicates that increased spermidine intake may decrease mortality, even when controlled for age, gender, caloric intake, and other variables. See, e.g., Kiechl S, et al. Higher spermidine intake is linked to lower mortality: a prospective population-based study, Am J Clin Nutr. 2018 08 1; 108(2):371-80.

Spermidine can be extracted from natural sources for inclusion in dietary supplements. The sources and extraction methods for producing such supplemental spermidine may impact a human body differently, however. The primary sources for purified spermidine are wheat germ and rice germ.

Several methods to extract spermidine from animals and plants have been disclosed. See, e.g., Chinese Patent CN112778138A and Chinese Pub. Nos. CN110279738, CN114751830, CN112544888. However, the disclosed methods may involve high cost, difficulties in scaling for industry, and/or are not compliant with food safety standards.

As a bio-polyamine, the molecule of spermidine may be weakly alkaline. The natural spermidine in wheat germ mostly exists as a salt such as oxalate or sulfate.

### SUMMARY

The present disclosure provides methods of purifying spermidine and compositions produced by said methods.

In one aspect, the present disclosure provides a method of extracting spermidine from rice germ, the method comprising:
(a) mixing rice germ with a first solvent to produce a first extraction solution;
(b) filtering the first extraction solution to produce a first filtrate; and
(c) concentrating the first filtrate to obtain a coarse extract of spermidine comprising less than about 1% by weight of spermidine;
(d) mixing the coarse extract of spermidine with a second solvent in the presence of citric acid to form a second extraction solution;
(e) filtering the second extraction solution to produce a second filtrate; and
(f) concentrating the second filtrate to obtain a spermidine extract composition comprising at least about 1% by weight of spermidine.

In another aspect, the present disclosure provides for a spermidine extract composition obtained from the method described herein, including about 1% to about 5% by weight or spermidine.

In another aspect, the present disclosure provides a method of preparing a high concentration spermidine extract, the method comprising:
mixing a coarse spermidine extract comprising less than 1% by weight of spermidine with a solvent comprising ethanol in the presence citric acid to form an extraction solution;
filtering the extraction solution to produce a filtrate; and
concentrating the filtrate to obtain a spermidine extract composition comprising at least 1% by weight of spermidine.

### BRIEF DESCRIPTION OF THE DRAWINGS

FIG. 1 illustrates a method of purifying spermidine from rice germ accordingly to certain embodiments of the present disclosure.

### DETAILED DESCRIPTION

The present disclosure relates to a novel high efficiency production method for extracting spermidine from rice germ. In some examples, the method includes: extracting spermidine from the rice germ with a first solvent containing ethanol to form a first extraction solution; filtering the first extraction solution; concentrating the filtrate to obtain a coarse extract; optionally mixing the coarse extract with citric acid and extracting with a second solvent containing ethanol to form a second extraction solution; filtering the second extraction solution to remove insoluble matter, and concentrating the filtrate under vacuum to obtain a rice germ extract with a spermidine content of at least about 1% by weight (e.g., about 1% to about 5% by weight). In some embodiments, the method described herein provides a total recovery rate of spermidine of at least about 50%, 60%, 70%, 80% or 90% from rice germ.

### Overview

Most spermidine on the market comes from wheat germ. A major difference between these wheat germ-purified spermidine and rice germ-purified spermidine is that spermidine extracted from wheat germ contains gluten, while that extracted from rice germ does not. For people with gluten intolerance or allergy, products containing wheat germ should be avoided as much as possible. Wheat germ products may also have negative effects such as increasing blood sugar on people with a low-carbohydrate diet, for example those having diabetes. Additionally, wheat germ extract may have some mild side effects, including diarrhea, nausea, bloating, and dizziness. Thus, for certain people, the spermidine extracted from rice germ may be more suitable than spermidine extracted from wheat. Especially for those with gluten intolerance, allergy, or on a low-carbohydrate diet, spermidine extracted from rice germ may be particularly suitable.

Generally, the content of spermidine in wheat germ and rice germ is between 100-300 ppm. Currently, the spermidine content in wheat germ-based spermidine extract on the market is usually around 0.1%-0.3% by weight. Such levels are inconvenient for use, as such concentrations may be relatively low in comparison dietary need of a user. The methods of the present disclosure provide an efficient method to extract spermidine from rice germ so that the spermidine concentration of a resulting spermidine extract could be 10-30 times higher than wheat germ-based spermidine. In accordance with the present disclosure, extracts sourced from rice germ may have a spermidine content of between about 1% to about 5% by weight.

Spermidine has been extracted from rice germ with citric acid solution as an extraction agent. However, when extracting spermidine from a large amount of gelatinized starch, this approach may lead to the difficulty in filtering the extraction and harsh production environment, which may be an issue for industrial scaling. The present disclosure provides for methods from rice germ with high efficiency that prevent or mitigate such scaling issues.

### Reducing Starch Gelatinization

Differential scanning calorimetry (DSC) may be used to assess regularity of starch in the rice germ gelatinizing in ethanol at different concentrations. As the concentration of ethanol increases, the enthalpy of gelatinization of starch may gradually decrease. When the concentration of ethanol is about 70% (v/v), the enthalpy of gelatinization of starch may be greatly reduced, which may indicate that the gelatinization and swelling of starch may be completely or substantially inhibited under at this ethanol concentration.

The present disclosure provides for extracting rice germ with a solvent comprising about 70% to about 85% ethanol (v/v). As the starch is not or substantially not gelatinized under these conditions, a filtration step may be more easily executed. Such an easy filtration step, in turn, improves the production environment and makes industrialization (e.g., scalability) easier. However, the recovery rate of spermidine extracted using about 70%-85% ethanol (v/v) from rice germ may be lower than that of water extraction where exposure times are just a few hours or less. The methods disclosed herein may increase recovery rate by including an extended extraction time of at least 6, 8, 10, or 12 hours. Such an extraction can increase the amount of spermidine recovered from rice germ to over 50%, 55%, 60%, 65%, 70%, 75%, 80%, 85% or 90%.

The content of spermidine in the extract may be further increased by increasing the concentration of ethanol in the extraction solvent. Alone, however, increasing the concentration of ethanol in the extraction solvent may reduce the recovery rate of spermidine from the rice germ. The present disclosure provides for use of citric acid, alongside high-concentration ethanol, to raise recovery rate. Citric acid may make the high-concentration ethanol extraction solvent slightly acidic, which may help increase spermidine recovery.

### Methods of Extracting Spermidine

One aspect of the present disclosure provides a method of extracting spermidine from rice germ, the method comprising:
(a) mixing rice germ with a first solvent to produce a first extraction solution;
(b) filtering the first extraction solution to produce a first filtrate; and
(c) concentrating the first filtrate to obtain a coarse extract of spermidine comprising less than about 1% by weight of spermidine;
(d) mixing the coarse extract of spermidine with a second solvent in the presence of citric acid to form a second extraction solution;
(e) filtering the second extraction solution to produce a second filtrate; and
(f) concentrating the second filtrate to obtain a spermidine extract composition comprising at least about 1% by weight of spermidine.

In some embodiments of the method described herein, the spermidine extract comprises about or at least about 1% to about 5% by weight of spermidine. For example, the spermidine extract comprises at least about 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9% or 5.0% spermidine, or a range defined by any two of the preceding values.

In some embodiments of the method described herein, the coarse extract of spermidine comprises about 0.1% to 0.9%, 0.2% to 0.8% or 0.3% to about 0.8% by weight of spermidine. For example, the coarse extract of spermidine comprises about 0.1%, 0.2%, 0.3%, 0.4%, 0.5%, 0.6%, 0.7%, 0.8% or 0.9% of spermidine.

In some embodiments of the method described herein, the citric acid is about 0.05% to about 5% by weight of the coarse extract of spermidine. For example, the citric acid is about 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9% or 5.0% by weight of the coarse extract of spermidine. In some further embodiments, the citric acid is about 1% to about 4% by weight of the coarse extract of spermidine.

In some embodiments of the method described herein, the first solvent comprises or is about 70% to about 85% ethanol (v/v). For example, the first solvent comprises or is about 70%, 75%, 80%, or 85% ethanol (v/v).

In some embodiments of the method described herein, the second solvent comprises or is about 70% to about 95% or about 85% to 95% ethanol (v/v). For example, the second solvent comprises or is about 70%, 75%, 80%, 85%, 90%, or 95% ethanol (v/v).

In some embodiments of the method described herein, the weight of the first solvent is about 4 to 10 times the weight of the rice germ, for example, about 4, 5, 6, 7, 8, 9 or 10 times of the weight of the rice germ.

In some embodiments of the method described herein, the weight of the second solvent is about 4 to 10 times the weight of the coarse extract of spermidine, for example, about 4, 5, 6, 7, 8, 9 or 10 times of the weight of the coarse extract of spermidine.

In some embodiments of the method described herein, in step (a) the rice germ is mixed with the first solvent at a temperature of about 60° C. to about 85° C. for at least 6, 8, 10 or 12 hours. In some further embodiments, in step (a) the rice germ is mixed with the first solvent at about 80° C. to about 82° C. for at least 6, 8, 10, 12, 14 or 16 hours.

In some embodiments of the method described herein, in step (d) the coarse extract of spermidine is mixed with the second solvent at a temperature about 60° C. to about 85° C. for at least 2, 3, 4, 5 or 6 hours.

In any embodiments of the method described herein, the rice germ comprises dried rice germ powder. In some further embodiments, the rice germ power comprises or is defatted rice germ power.

In any embodiments of the method described herein, the total extraction recovery rate of spermidine from the rice germ is at least about 50%, 55%, 60%, 65%, 70%, 75%, 80%, 85% or 90%.

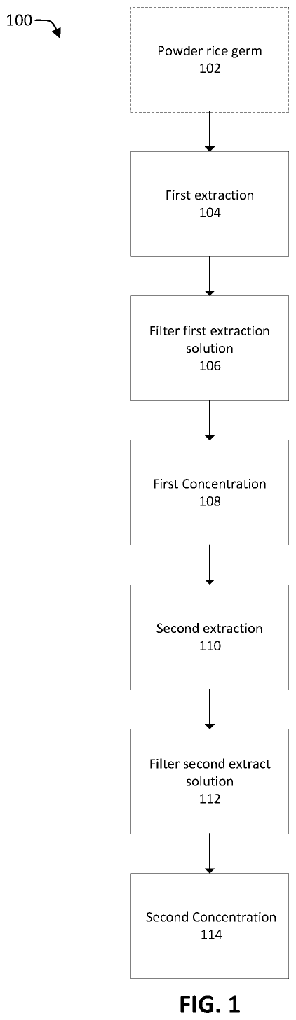

FIG. 1 illustrates an example process 100 for extracting spermidine from rice germ in accordance with the present disclosure.

At step 102, the rice germ is rice germ powder. Rice germ may be dried and crushed into a powder to facilitate subsequent extraction of spermidine using a solvent. The rice germ powder may include defatted rice germ powder.

At step 104, the first extraction is carried out. In some examples, the spermidine source (e.g., the rice germ powder) is mixed with a first solvent to extract the spermidine, producing a first extraction solution. In some examples, the first solvent includes 70% to 85% ethanol (v/v). In some examples, the first solvent includes 80% to 85% ethanol (v/v). In some examples, the weight of the first solvent used in step 104 may be 4 times to 10 times the weight of the spermidine source. In some examples, the step 104 may be carried out for at least 6, 8, 10, or 12 hours. In some examples, the step 104 may be carried out for at least 12, 14 or 16 hours. In some examples, the step 104 may be carried out at a temperature of about 60° C. to about 85° C. In some examples, the step 104 may be carried out at a temperature of about 80° C. to about 82° C.

At step 106, filter the first extraction solution to create a first filtrate. In some examples, the extraction solution may be cooled to room temperature prior to filtration.

At step 108, concentrate the first filtrate to produce a coarse extract. In some examples, step 108 may include heating the first filtrate. In some examples, the first filtrate may be concentrated under vacuum. In some examples, step 108 may produce a course extract having a spermidine concentration of 0.3% to 0.8% by weight. In some examples, step 108 may produce a course extract having a spermidine concentration of 0.5% to 0.8% by weight.

At step 110, carry out the second extraction. In some examples, the coarse extract is exposed to a second solvent to create a second extraction solution. In some examples, citric acid may be added to the second extraction solution. In some further examples, 0.05% to 5% by weight of citric acid is added to the second extraction solution. In some examples, the second solvent includes 85% to 95% ethanol (v/v). In some examples, the weight of the second solvent used in step 104 may be 4 times to 10 times the weight of the coarse extract.

At step 112, filter the second extraction solution to produce a second filtrate. In some examples, the second extraction solution may be cooled to room temperature prior to filtration.

At step 114, concentrate the second filtrate. The second filtrate may be concentrated under vacuum. The step 114 may produce a spermidine extract comprising a spermidine content of at least 1.0% to 5.0% by weight. In some examples, the step 114 may produce a spermidine extract comprising a spermidine content of 1.0%, 1.1%, 1.2%, 1.3%, 4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, or 5.0% by weight, or in a range defined by any two of the preceding values. In some examples, the spermidine extract may comprise a spermidine content of 2% to 3% by weight. In some examples, the total extraction recovery rate of spermidine from the rice germ is at least about 50%, 55%, 60%, 65%, 70%, 75%, 80%, 85% or 90%.

In some embodiments, as a preliminary step, rice germ can be exposed to about 70% to about 85% ethanol (v/v), where the weight of the ethanol is about 4-10 times the weight of rice germ or spermidine source. The temperature during such exposure may be between 60° C.-85° C., and, as mentioned above, the exposure may occur for least 12 hours. In some embodiments, the extraction solution can be cooled to room temperature and filtered. The filtrate can be heated and vacuum concentrated to obtain a coarse extract of spermidine with a spermidine content of 0.3%-0.8%. Such a coarse extract may recover over 90% of the spermidine present in the rice germ.

In some examples, the present disclosure provides for addition of citric acid at 0.05% to 5% by weight of the coarse extract of spermidine which then being extracted with a concentration of 85% to 95% ethanol 4 to 10 times the mass of the coarse extract of spermidine under a temperature of 60° C. to 85° C. for at least 6 hours, and then cools the extract to room temperature and filters out the insoluble materials. In some examples, the filtrate is vacuum concentrated to obtain a rice germ extract with a spermidine content of 1.0% to 5.0%, and the total recovery rate of spermidine can reach higher than 80%.

In some examples, a method for efficiently extracting spermidine from rice germ can use defatted rice germ powder as a source of spermidine. In some examples, 80% to 85% ethanol (v/v) may be used as the extraction solvent. In some examples, the extraction solvent mass used is about 8 times the mass of the rice germ used. The preferred extraction temperature is 80° C. to 82° C., and the preferred extraction time is more than 16 hours. After cooling the extract to room temperature and filtering out the insoluble materials, the filtrate is concentrated to obtain crude spermidine paste with a spermidine content of 0.5% to 0.8%.

### Methods of Preparing a High Concentration Spermidine Extract

Another aspect of the present disclosure relates to a method of preparing a high concentration spermidine extract, the method comprising:
mixing a coarse spermidine extract comprising less than 1% by weight of spermidine with a solvent comprising ethanol in the presence citric acid to form an extraction solution;
filtering the extraction solution to produce a filtrate; and
concentrating the filtrate to obtain a spermidine extract composition comprising at least 1% by weight of spermidine.

In some embodiments of the method described herein, the spermidine extract composition comprises about or at least about 1% to about 5% by weight of spermidine. For example, the spermidine extract comprises at least about 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9% or 5.0% spermidine, or a range defined by any two of the preceding values. In some embodiments, the citric acid is about 0.05% to about 5% by weight of the coarse spermidine extract. For example, the citric acid is about 0.05%, 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9% or 5.0% by weight of the coarse extract of spermidine. In some further embodiments, the citric acid is about 1% to about 4% by weight of the coarse spermidine extract. In some embodiments, the coarse spermidine extract is mixed with about 70% to about 85% (v/v) ethanol at a temperature of about 60° C. to about 85° C.

As described herein, a production method for efficiently extracting high spermidine content from rice germ may include adding citric acid to the crude spermidine paste (also referred to herein as a course extract). In some examples, the citric acid addition amount may be 0.05% to 5% of the mass of the crude spermidine paste, using a mass multiple of 4 to 10 times the mass of the crude spermidine paste. In such examples, a concentration of 85% to 95% ethanol may be used as a extraction solvent. In such examples, the extraction may include heating the mixture to 60° C. to 85° C. and extracting for at least 6 hours. In some examples, the citric acid addition amount is 2% to 4% of the mass of the crude spermidine paste. In some examples, the extraction solvent mass is 6 to 8 times the mass of the crude spermidine paste. In some examples, the extraction solvent is 93% to 95% ethanol (v/v). In some examples, the extraction is carried out at a temperature of about 80° C. to about 82° C. In some examples, the extraction lasts 12 hours or more. In some examples, the extraction solution may be cooled to room temperature and filtered to remove insoluble materials, producing a filtrate. In some examples, the filtrate is vacuum concentrated to obtain an extract with a spermidine content of at least about 1% to about 5%.

### Compositions of Spermidine Extracts

The present disclosure provides for compositions of spermidine extract produced by the methods discussed herein. In some examples, such composition includes a spermidine content from about or at least about 1% to about 5% by weight. Optionally, the composition may contain a small amount of citric acid. In some embodiments, the composition may be slightly acidic. For example, the composition may have a pH from about 5 to about 6.8. In some further examples, the composition is gluten-free or essentially gluten free. As one of the criteria for using the term “gluten-free,” the FDA set a limit of less than 20 ppm (parts per million). That is the lowest level that can be consistently detected in foods using valid scientific analytical tools. In some further examples, the composition comprises 0.1% to 1.0% by weight of spermine. In some examples, the composition comprises 0.1% to 0.8% of putrescine.

### EXAMPLES

Various aspects of the present disclosure are described in detail in conjunction with the following examples. Such examples are provided for illustrative purposes and are not intended to be an extensive list of all possible implementations in accordance with the present disclosure. Based on the examples herein, all other implementation examples obtained by those skilled in the art without creative labor are within the scope of the present disclosure.

### Example 1—Production of Course Extract

Defatted rice germ was crushed into powder. 90 kg of 85% ethanol (v/v) was added to 10 kg of the rice germ powder. The mixture was heated to about 80° C. and stirred for 16 hours to extract. Under these conditions, the extract did not gelatinized, and thus the extract was easily filtered after cooling to room temperature. After removing the insoluble matter by filtration, 307 g of rough rice germ paste was obtained by concentrating the filtrate. The content of spermidine in the rough rice germ paste was measured to be 0.60%, and the recovery rate of spermidine extracted from rice germ was 92.1%.

### Example 2—Production of Course Extract

Rice germ was crushed into powder. 80 kg of 70% ethanol was added to 10 kg of the rice germ powder. The mixture was heated to about 82° C. and stirred for 24 hours to extract. Under these conditions, the extract did not gelatinized, and thus the extract was easy to filter after cooling to room temperature. After removing the insoluble matter by filtration, 357 g of rough rice germ paste was obtained by concentrating the filtrate. The content of spermidine in the rough rice germ paste was measured to be 0.51%, and the recovery rate of spermidine extracted from rice germ was 91.0%.

### Example 3—Production of Spermidine Extract Using Citric Acid

Rice germ was crushed into powder. 80 kg of 80% ethanol was added to 10 kg of rice germ powder. The mixture had been heated to about 82° C. and stirred for 18 hours to extract. Under these conditions, the extract was not gelatinized, and the extract was thus easily filtered after cooling to room temperature. After removing the insoluble matter by filtration, 348 g of rough rice germ paste was obtained by concentrating the filtrate. The content of spermidine in the rough rice germ paste was detected to be 0.53%. 10 g of citric acid was added to the 348 g of spermidine rough paste, and 2 kg of 95% ethanol was used to stir and extract at 80° C. for 12 hours. Under these conditions, the extract was not gelatinized, and the extract was thus easy to filter after cooling to room temperature. After removing the insoluble matter by filtration, the filtrate was vacuum concentrated and 71 g of rice germ extract was finally obtained. The content of spermidine in the rice germ extract was 2.33%, and the total recovery rate of spermidine extracted from rice germ was 82.7%.

### Example 4—Measured for Spermidine, Spermine, and Putrescine Content

Defatted rice germ was crushed into powder. 4000 kg of 80% ethanol was added to 500 kg of the defatted rice germ powder. The mixture was heated to around 82° C. and stirred for 36 hours to extract. The extract did not gelatinize and was easy to filter after cooling to room temperature and removing insoluble components of the mixture. The filtered liquid was concentrated to obtain 13.8 kg of crude rice germ extract. The crude extract was measured to have a spermidine content of 0.67%. 600 g of citric acid was added to the 13.8 kg crude spermidine extract. 85 kg of 93% ethanol was added and stirred at 81° C. for 24 hours to extract. The extract did not gelatinize and was easy to filter after cooling to room temperature and removing insoluble components of the mixture. The filtered liquid was heated and vacuum concentrated to obtain 2.94 kg of rice germ extract. The rice germ extract was tested to have a spermidine content of 2.82%, a spermine content of 0.55%, and a putrescine content of 0.28%. The total extraction recovery rate of spermidine in rice germ was 83.1%.

### Example 5—Production of Spermidine Extract Using Citric Acid

Rice germ was crushed into powder. 4000 kg of 85% ethanol (v/v) was added to 500 kg of the rice germ powder, the mixture was heated to about 82° C. The mixture was stirred for 24 hours to allow for extraction of spermidine from the rice germ. Under these conditions, the extract did not gelatinize, and was easily filtered after cooling to room temperature. After removing the insoluble material by filtration, a crude rice germ paste of 12.7 kg was obtained by concentrating the filtrate. The crude germ paste was tested and found to contain 0.73% spermidine. 500 g of citric acid was added to the 12.7 kg crude spermidine paste, and the mixture was extracted by stirring with 90 kg of 95% ethanol at 81° C. for 20 hours. The extract did not gelatinize and was easily filtered after cooling to room temperature. After removing the insoluble material by filtration, the filtrate was vacuum concentrated and ultimately yielded 3.28 kg of rice germ extract. The spermidine content of the extract was found to be 2.55%, and the total extraction recovery of spermidine from rice germ was 83.6%.

### Example 6—Low Ethanol Concentration

Defatted rice germ was crushed into powder. 80 kg of 40% ethanol was added to 10 kg of the defatted rice germ powder. The mixture was heated to about 80° C. and stirred for 16 hours. Under these conditions, the extract gelatinized, and, after cooling to room temperature, was difficult to filter. After removing the insoluble material by filtration, a crude rice germ paste of 1.13 kg was obtained by concentrating the filtrate. The crude paste was tested and found to contain 0.11% spermidine. The spermidine extraction recovery from rice germ was 62.2%.

### Example 7—No Addition of Citric Acid

Rice germ was crushed into powder. 80 kg of 80% ethanol was added to 10 kg of rice germ powder. The mixture was heated to about 82° C. and stirred for 18 hours. Under these conditions, the extract did not gelatinize, and was easily filtered after cooling to room temperature. After removing the insoluble material by filtration, a crude rice germ paste of 339 g was obtained by concentrating the filtrate. The crude paste was tested and found to contain 0.54% spermidine. 2 kg of 95% ethanol was added to the 339 g crude spermidine paste, and the mixture was extracted by stirring at 80° C. for 24 hours without the addition of citric acid. The extract did not gelatinize, and after cooling to room temperature, it was easily filtered. After removing the insoluble material by filtration, the filtrate was vacuum concentrated and ultimately yielded 143 g of rice germ extract. The spermidine content of the extract was found to be 0.89%, and the total extraction recovery of spermidine from rice germ was 63.8%.

### Terminology

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise. As used in the specification and the appended claims, the singular forms “a.” “an” and “the” include plural referents unless the context clearly dictates otherwise. Unless otherwise indicated, conventional methods of mass spectroscopy, NMR, and HPLC are employed. The use of “or” or “and” means “and/or” unless stated otherwise. Furthermore, use of the term “including” as well as other forms, such as “include”, “includes,” and “included,” is not limiting.

While the disclosure has been illustrated and described in detail in the foregoing description, such description is to be considered illustrative or exemplary and not restrictive. The disclosure is not limited to the disclosed embodiments. Variations to the disclosed embodiments can be understood and effected by those skilled in the art in practicing the claimed disclosure, from a study of the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated.

Where a range of values is provided, it is understood that the upper and lower limit, and each intervening value between the upper and lower limit of the range is encompassed within the embodiments.

As used herein, “recovery rate” is the proportion of a component that is isolated from a raw material containing that component divided by the total amount of that component originally present in the raw material. For example, “extraction recovery rate of spermidine from the rice germ” refers to the proportion of (spermidine isolated from rice germ/spermidine originally present in the rice germ). Recovery rate can be used to describe a single step of an extraction process, several steps of an extraction process, or an extraction process as a whole.

Embodiments have been described in connection with the accompanying drawings. However, In addition, the foregoing embodiments have been described at a level of detail to allow one of ordinary skill in the art to make and use the devices, systems, methods, etc. described herein. A wide variety of variation is possible. Components, elements, and/or steps can be altered, added, removed, or rearranged. While certain embodiments have been explicitly described, other embodiments will become apparent to those of ordinary skill in the art based on this disclosure.

Conditional language used herein, for example, “can,” “could.” “might,” “may.” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements, and/or states are in any way required for one or more embodiments or that one or more embodiments necessarily include logic for deciding, with or without author input or prompting, whether these features, elements and/or states are included or are to be performed in any particular embodiment.

Conjunctive language such as the phrase “at least one of X, Y, and Z.” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

It is to be understood that the ranges provided herein include the stated range and any value or sub-range within the stated range, as if such value or sub-range were explicitly recited. For example, a range from about 60° C. to about 85° C. should be interpreted to include not only the explicitly recited limits of from about 60° C. to about 85° C., but also to include individual values, such as about 80.5° C., about 68° C., about 61.2° C., etc., and sub-ranges, such as from about 75° C. to about 80° C., etc.

Depending on the embodiment, certain acts, events, or functions of any of the methods described herein can be performed in a different sequence, can be added, merged, or left out altogether (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, engines, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

While the above detailed description has shown, described, and pointed out novel features as applied to various embodiments, it will be understood that various omissions, substitutions, and changes in the form and details of the methods and compositions illustrated can be made without departing from the spirit of the disclosure. As will be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A method of extracting spermidine from rice germ, the method comprising:
(a) mixing rice germ with a first solvent to produce a first extraction solution;
(b) filtering the first extraction solution to produce a first filtrate; and
(c) concentrating the first filtrate to obtain a coarse extract of spermidine comprising less than about 1% by weight of spermidine;
(d) mixing the coarse extract of spermidine with a second solvent in the presence of citric acid to form a second extraction solution;
(e) filtering the second extraction solution to produce a second filtrate; and
(f) concentrating the second filtrate to obtain a spermidine extract composition comprising at least about 1% by weight of spermidine.

2. The method of claim 1, wherein the spermidine extract comprises from about 1% to about 5% by weight of spermidine.

3. The method of claim 1, wherein the coarse extract of spermidine comprises about 0.3% to about 0.8% by weight spermidine.

4. The method of claim 1, wherein the citric acid is about 0.05% to about 5% by weight of the coarse extract of spermidine.

5. The method of claim 4, wherein the citric acid is about 1% to about 4% by weight of the coarse extract of spermidine.

6. The method of claim 1, wherein the first solvent comprises about 70% to about 85% ethanol (v/v).

7. The method of claim 1, wherein the second solvent comprises about 85% to about 95% ethanol (v/v).

8. The method of claim 1, wherein the weight of the first solvent is about 4 to 10 times the weight of the rice germ.

9. The method of claim 1, wherein the weight of the second solvent is about 4 to 10 times the weight of the coarse extract of spermidine.

10. The method of claim 1, wherein in step (a) the rice germ is mixed with the first solvent at a temperature of about 60° C. to about 85° C. for at least 12 hours.

11. The method of claim 10, wherein in step (a) the rice germ is mixed with the first solvent at about 80° C. to about 82° C. for at least 16 hours.

12. The method of claim 1, wherein in step (d) the coarse extract of spermidine is mixed with the second solvent at a temperature about 60° C. to about 85° C. for at least 6 hours.

13. The method of claim 1, wherein the rice germ comprises dried rice germ powder.

14. The method of claim 1, wherein the total extraction recovery rate of spermidine from the rice germ is at least about 50%.

15. A spermidine extract composition prepared by the method of claim 1, comprising about 1% to about 5% by weight or spermidine.

16. The spermidine extract composition of claim 15, wherein the composition is essentially gluten free.

17. The spermidine extract composition of claim 15, wherein the composition comprises about 0.1% to about 0.8% of putrescine.

18. A method of preparing a high concentration spermidine extract, the method comprising:
mixing a coarse spermidine extract comprising less than 1% by weight of spermidine with a solvent comprising ethanol in the presence citric acid to form an extraction solution;
filtering the extraction solution to produce a filtrate; and
concentrating the filtrate to obtain a spermidine extract composition comprising at least 1% by weight of spermidine.

19. The method of claim 18, wherein the citric acid is about 0.05% to about 5% by weight of the coarse spermidine extract.

20. The method of claim 18, wherein the coarse spermidine extract is mixed with about 70% to about 85% (v/v) ethanol at a temperature of about 60° C. to about 85° C.
